(19) United States
(12) Patent Application Publication
(10) Pub. No.: US 2003/0221903 A1
(43) Pub. Date: Dec. 4, 2003

(54) Indicia bearing and color-coded medical device and associated methods

(76) Inventors: Mark William Roby, Lake Mary, FL (US); Saundra Lynn Roby, Lake Mary, FL (US)

Correspondence Address: Jeffrey S. Whittle, BRACEWELL & PATTERSON, L.L.P., P.O. Box 61389, Houston, TX, 77208-1389, US

(21) Appl. No.: 10/156,452

(22) Filed: May 28, 2002

(51) Int. Cl. A61B 7/02

(52) U.S. Cl. 181/131; 181/128

(57) ABSTRACT

A stethoscope assembly for sports enthusiasts is provided which preferably includes at least one sound receiver assembly, a tube assembly having a proximal end portion connected to the at least one sound receiver assembly and extending outwardly therefrom toward the user, a first and second earpieces each having a proximal end portion connected to a distal end portion of the tube assembly and extending outwardly therefrom toward the user. The tube assembly further preferably is visibly colored with at least one of a first color associated with a sports team to thereby define a first sports team color and a second contrasting color also associated with the sports team to thereby define a second sports team color.

### FIELD OF THE INVENTION

The present invention relates to the field of medical devices and, more particularly, to stethoscope assemblies for sports enthusiasts and methods for retrofitting existing stethoscopes for sports stethoscopes as well as kits for converting existing stethoscopes to sports stethoscopes.

### BACKGROUND OF THE INVENTION

A stethoscope is an essential diagnostic instrument used in daily medical practice. In the clinical setting, the stethoscope is used as a listening device to evaluate heart, respiratory, pleural, uterine, intestinal and other physiological sound which may be conveyed to the user. A stethoscope generally includes a sound receiver assembly, which is acoustically connected to an earpiece assembly. The sound receiver assembly has a transducer diaphragm, usually a membrane-like partitioning surface, pressed firmly on a body surface of a patient. The earpiece assembly has two earpieces (i.e., sound transmission members). The sound monitored by the transducer diaphragm is generally surface sound which is transmitted from various locations on the body surface through the diaphragm to the earpiece assembly. Common stethoscope designs acoustically connect the diaphragm with the earpieces with a tube assembly for the acoustic passage of the monitored surface sound.

One of the common and practical problems with conventional stethoscopes is patient apprehension of the instrument before and during its application to the patient's body parts. Solutions to such problem include applying indicia that is appealing to a patient to a transducer diaphragm to help to create a comfortable setting for the patient. It is believed that providing appealing indicia, such as illustrations or graphical designs, on a stethoscope's transducer diaphragm outer surface may serve to at least partially pacify such an apprehensive patient prior to applying that surface to the patient's body. It is also believed that some patients may be further relieved of apprehension by providing a variety of such indicia or designs on interchangeable transducer diaphragms so that a particular indicia may be chosen for or by a given patient. An example of such stethoscope device can be seen in U.S. Pat. No. 5,920,038 by Foster titled “Stethoscope Device Assembly.”

Some other known stethoscopes use stethoscope covers which are designed to be temporarily secured onto the sound receiver assembly of the stethoscope for the intended purpose of addressing concerns at the diaphragm-patient interface when the diaphragm is pressed against the body surface as well as for pacifying an apprehensive patient. Examples of such stethoscope devices can be seen in U.S. Pat. No. Des. 422,701 by Gallagher titled “Stethoscope Diaphragm Cover,” U.S. Pat. No. Des. 422,701 by Gallagher titled “Stethoscope Diaphragm Cove,” and U.S. Pat. No. Des. 431,652 by Cherney titled “Novelty Stethoscope Attachment Kit.” Still some other known stethoscopes use special shape designs for the sound receiver assembly for improved acoustic detection and transmission. An example of such a stethoscope can be seen in U.S. Pat. No. 5,420,382 by Katz titled “Sea-Shell Stethoscope Head.”

### SUMMARY OF THE INVENTION

With the foregoing in mind, the present invention advantageously provides several stethoscope assemblies that are specifically designed for sports enthusiasts and methods for retrofitting the existing stethoscopes for sports stethoscopes. The present invention also advantageously provides kits for converting existing stethoscopes to sports stethoscopes. Additionally, the present invention advantageously provides medical personnel a momento of a school sports team they attended or of which they are a fan, of a sports team for whom they are a fan, and as a reminder or symbol of collegiate memories, loyalties, or friendships. Further, the present invention provides an advertisement vehicle for sports team. Still further, the present invention advantageously provides sports enthusiasts or others methods to easily retrofit existing medical devices with sports indicia to indicate endorsements, loyalties, or the like.

According to an embodiment of the present invention, a dual channel stethoscope assembly for sports enthusiasts is provided which has at least one sound receiver assembly, first and second tubes each having a proximal end portion connected to the at least one sound receiver assembly and extending outwardly therefrom toward the user, at least one tube retainer retaining at least a portion of each of the first and second tubes in a position closely adjacent each other, first and second earpieces each having a proximal end portion connected to a distal end portion of each of the first and second tubes and extending outwardly therefrom toward the user, and first and second eartips each connected to a distal end portion of each of the first and second earpieces and adapted to be positioned closely adjacent the user's ears.

The at least one sound receiver assembly preferably includes a bell, a retaining ring mounted to the bell, and a transducer diaphragm mounted to the bell, retained in position by the retaining ring, and being adapted to abuttingly contact the body surface to thereby receive sound therefrom. The first and second tubes preferably are visibly colored with at least one of a first color associated with a sports team to thereby define the first sports team color and a second contrasting color also associated with the sports team to thereby define a second sports team color.

Preferably, the sports team may include at least one of the following: a school sports team, a professional sports team and a club sports team. To further target sports teams, the first and second tubes may have sports indicia positioned thereon. In the present invention, for example, the sports indicia have colors corresponding to the first and second sports team colors and may include sports logos, sponsor logos, licensed logos, character's logos, and other famous indicia readily recognizable by both the user and the patient. Representative examples of the sports indicia include college, high school, graduate school, professional, club, civic or community, church and other sports teams' logos. Still preferably, the first and second earpieces may be visibly colored with at least one of the first and second sports team colors. For example, the first earpiece can be colored with the first sports team color, e.g. blue, and the second earpiece with the second sports team color, e.g., white or orange. The bell and/or the retaining ring can also be colored with at least one of the first and second sports team colors. Furthermore, the bell can have a shape substantially similar to a ball associated with the sports team, e.g. basketball, soccer ball, football, volleyball, baseball. Still preferably, the transducer diaphragm and/or the at least one tube retainer can further have the sports indicia positioned thereon.

According to another embodiment of the present invention, a single channel stethoscope for sports enthusiasts preferably includes at least one sound receiver assembly, a tube having a proximal end portion connected to and having hollow portions in fluid communication with the at least one sound receiver assembly and extending outwardly from the at least one sound receiver assembly toward the user and a manifold connected to a distal end portion of the tube, in fluid communication with the hollow portions of the tube, and extending outwardly from the distal end portion of the tube. The manifold preferably includes at least first and second hollow portions each in fluid communication with the hollow portions of the tube. The stethoscope preferably further includes first and second earpieces each having a proximal end portion connected to the manifold, in fluid communication with each of the at least the first and second hollow portions of the manifold, and extending outwardly toward the user, and first and second eartips each connected to a distal end portion of each of the first and second earpieces and adapted to be positioned closely adjacent the user's ears.

The at least one sound receiver assembly preferably includes a bell, a retaining ring mounted to the bell, and a transducer diaphragm mounted to the bell, retained in position by the retaining ring, and being adapted to abuttingly contact the body surface to thereby receive sound therefrom. The tube and the manifold preferably are visibly colored with at least one of a first color associated with a sports team to thereby define the first sports team color and a second contrasting color also associated with the sports team to thereby define a second sports team color.

More particularly, the stethoscope assembly advantageously includes at least one sound receiver assembly, a tube assembly having a proximal end portion connected to the at least one sound receiver assembly and extending outwardly therefrom toward the user, and first and second earpieces each having a proximal end portion connected to a distal end portion of the tube assembly and extending outwardly therefrom toward the user. The tube assembly preferably is visibly colored with at least one of a first color associated with a sports team to thereby define a first sports team color and a second contrasting color also associated with the sports team to thereby define a second sports team color.

The at least one sound receiver assembly preferably includes a bell, and a transducer diaphragm mounted to the bell and being adapted to abuttingly contact the body surface to thereby receive sound therefrom.

According to yet another aspect of the present invention, a kit is provided to convert an existing stethoscope to a sports enthusiast's stethoscope. The kit preferably has a container, a tube assembly positioned in the container and being visibly colored with at least one of a first color associated with a sports team to thereby define a first sports team color and a second contrasting color also associated with the sports team to thereby define a second sports team color, and at least first and second eartips positioned in the container and each having at least one of the first and second sports team colors associated therewith. Preferably, such kit can further have at least one transducer diaphragm positioned in the container and having a surface visibly colored with at least one of the first and second sports team colors.

According to still yet another aspect of the present invention, a kit is provided for converting an existing dual channel stethoscope to a sports enthusiast's dual channel stethoscope. The kit preferably includes a container, at least first and second tubes each positioned in the container and visibly colored with at least one of a first color associated with a sports team to thereby define a first sports team color and a second contrasting color also associated with the sports team to thereby define second sports team color, at least first and second eartips each positioned in the container and having at least one of the first and second sports team colors, and at least one transducer diaphragm positioned in the container and having a surface visibly colored with at least one of the first and second sports team colors.

Preferably, the sports team may include at least one of the following: a school sports team, a professional sports team and a club sports team. To further target sports teams the first and second tubes can have sports indicia positioned thereon. In the present invention, the sports indicia have colors corresponding to the first and second sports team colors and may include sports logos and other related sports indicia readily recognizable by both the user and the patient. Still preferably, the first and second eartips can be visibly colored with at least one of the first and second sports team colors. Still preferably, the at least one transducer diaphragm can further have sports indicia positioned thereon.

According to yet another aspect of the present invention, a kit is provided for converting an existing single channel stethoscope to a sports enthusiast's single channel stethoscope. The kit includes a container, at least one tube positioned in the container and being visibly colored with at least one of a first color associated with a sports team to thereby define a first sports team color and a second contrasting color also associated with the sports team to thereby define a second sports team color, at least one manifold positioned in the container and being visibly colored with at least one of the first and second sports team colors, at least first and second eartips positioned in the container and each having at least one of the first and second sports team colors associated therewith, and at least one transducer diaphragm positioned in the container and having a surface visibly colored with at least one of the first and second sports team colors.

The present invention further advantageously provides a method of retrofitting an existing stethoscope for a sports stethoscope. Such method preferably includes replacing a tube assembly of the existing stethoscope with a different tube assembly being visibly colored with at least one of a first color associated with a sports team to thereby define a first sports team color and a second contrasting color also associated with the sports team to thereby define a second sports team color.

Alternatively, the retrofitting method can further include replacing first and second eartips of the existing stethoscope with third and fourth eartips. Each of the third and fourth eartips preferably is visibly colored with at least one of the first and second sports team colors.

Still alternatively, the retrofitting method can further include replacing first and second earpieces of the existing stethoscope with third and fourth earpieces. Each of the third and fourth earpieces preferably is visibly colored with at least one of the first and second sports team colors.

Still alternatively, the retrofitting method can further include replacing at least one transducer diaphragm of the existing stethoscope with at least one transducer diaphragm being visibly colored with at least one of the first and second sports team colors.

To further target sports teams, the method can yet further include positioning sports indicia on at least one transducer diaphragm of the existing stethoscope, wherein the sports indicia have colors corresponding to the first and second sports team colors associated with the tube assembly. Further more, the method can include positioning the sports indicia on the tube assembly visibly colored with at least one of the first and second sports team colors. In the present invention, the sports indicia have colors corresponding to the first and second sports team colors and may include sports logos, and other sports related indicia readily recognizable by both the user and the patient. For dual channel stethoscope, the sports indicia may also be positioned on at least one tube retainer of the existing stethoscope.

According to yet another embodiment of the present invention, a method of doing business is advantageously provided by obtaining a license from at least one sports teams to use sports team identifiers associated with the sports team in association with a stethoscope, positioning sports team identifiers on a stethoscope so that a potential user of the stethoscope readily recognizes that the stethoscope is associated with the sports team, selling the stethoscope to the potential user, and distributing the stethoscope to the potential user after purchase of the stethoscope. Such method can further include advertising the stethoscope associated with the sports team.

Preferably, the sports team can include at least one of the following: a school sports team, a professional sports team and a club sports team. Still preferably, the sports identifier is positioned on at least one of the following: a tube assembly, first and second eartips, first and second earpieces, and a sound receiver assembly of the stethoscope.

Another method of doing business according to the present invention preferably includes obtaining a license from each of a plurality of different sports teams to use sports team identifiers associated with each of the plurality of sports teams in association with a plurality of stethoscopes; and manufacturing a plurality of stethoscopes. Each of the plurality of stethoscopes preferably has a different sports team identifier associated therewith. Such method can further include advertising each of the plurality of stethoscopes associated with each of the plurality of sports teams or selling the plurality of stethoscopes to the potential user and distributing the plurality of stethoscopes to the potential user after purchase of the stethoscopes.

Preferably, the sports team can include at least one of the following: a school sports team, a professional sports team and a club sports team. Still preferably, the sports identifier is positioned on at least one of the following: a tube assembly, first and second eartips, first and second earpieces, and a sound receiver assembly of the stethoscope.

Therefore, a stethoscope assembly for sports enthusiasts and methods/kits of retrofitting an existing stethoscope for sports stethoscope advantageously create a sports medical product or device for sports enthusiasts as well as pacifies an apprehensive patient by assisting medical personnel in focusing the patient on the device rather than the procedure or check up being performed.

### BRIEF DESCRIPTION OF THE DRAWING

Some of the features, advantages, and benefits of the present invention having been stated, others will become apparent as the description proceeds when taken in conjunction with the accompanying drawings in which:

FIG. 1 is a perspective view of three dual channel stethoscope assemblies each having different sports team colors and sports team indicia associated therewith according to an embodiment of the present invention;

FIG. 2 is a table illustrating five exemplary sports teams and corresponding color-coding of various parts of a stethoscope according to the present inventions;

FIG. 3A is a front elevational view of a dual channel stethoscope assembly according to an embodiment of the present invention;

FIG. 3B is an enlarged fragmentary elevational view of a tube retainer of a stethoscope assembly shown in FIG. 3A according to the present invention;

FIG. 3C is an exploded perspective view of the sound receiver assembly shown in FIG. 3A;

FIG. 4A is an enlarged fragmentary perspective view of a dual channel stethoscope assembly according to an embodiment of the present invention;

FIG. 4B is a fragmentary perspective view of a sound receiver assembly having the shape of a sports ball according to an embodiment of the present invention;

FIG. 5A is a front elevational view of a single channel stethoscope assembly according to an embodiment of the present invention;

FIG. 5B is a fragmentary perspective view of a single channel stethoscope assembly according to another embodiment of the present invention; and

FIG. 6 is a perspective view of a kit for converting an existing dual channel stethoscope to a sports stethoscope according to an embodiment of the present invention.

### DETAILED DESCRIPTION OF THE INVENTION

The present invention will now be described more fully hereinafter with reference to the accompanying drawings which illustrated preferred embodiments of the invention. This invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art. Like numbers refer to like elements throughout, and prime and/or double prime notation are used to indicate similar elements in alternative embodiments.

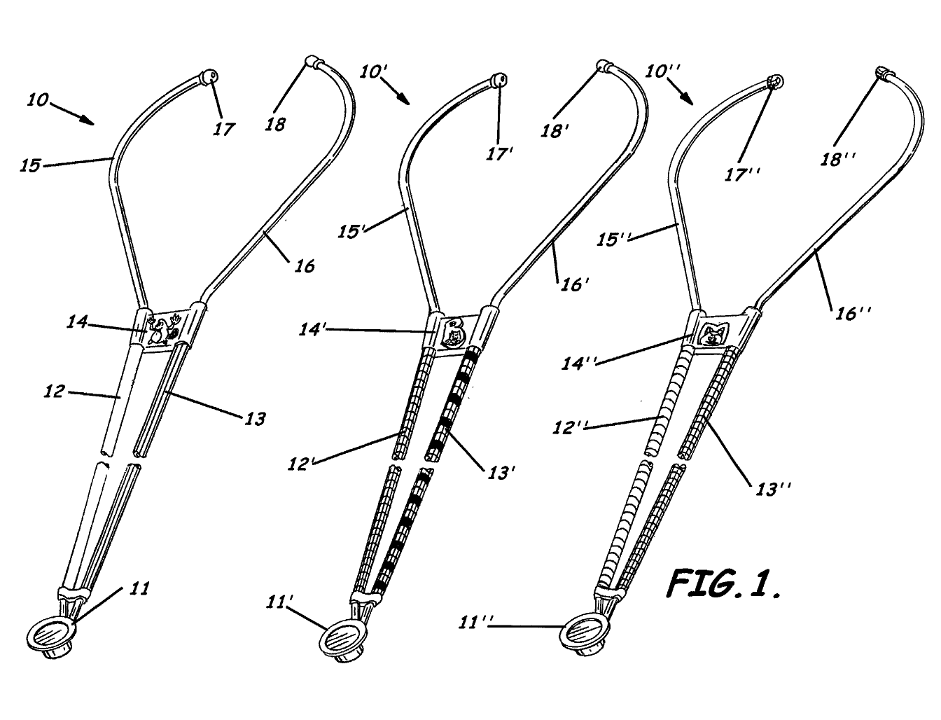

FIG. 1 illustrates three dual channel stethoscope assemblies 10, 10′, and 10″ for sports enthusiasts according to an embodiment of the present invention. The sports stethoscopes are used for monitoring sound by a user on a body surface of a patient when a proximal portion thereof is positioned closely adjacent the patient and a distal portion is positioned closely adjacent the user. The dual channel stethoscope assemblies 10, 10′, 10″ each preferably has a sound receiver assembly 11, 11′, 11″, a first tube 12, 12′, 12″ and a second tube 13, 13′, 13″ each having a proximal end portion connected to the sound receiver assembly 11, 11′, 11″ and extending outwardly therefrom toward the user, a tube retainer 14, 14′, 14″ retaining at least a portion of each of the first tube 12, 12′, 12″ and the second tube 13, 13′, 13″ in a position closely adjacent each other, a first earpiece 15, 15′, 15″ having a proximal end portion connected to a distal end portion of the first tube 12, 12′, 12″ and extending outwardly therefrom toward the user, a second earpiece 16, 16′, 16″ having a proximal end portion connected to a distal end portion of the second tube 13, 13′, ₁₃″ and extending outwardly therefrom toward the user, a first eartip 17, 17′, 17″ connected to a distal end portion of the first earpiece 15, 15′, 15″ and adapted to be positioned closely adjacent the user's first ear, and a second eartip 18, 18′, 18″ connected to a distal end portion of the second earpiece 16, 16′, 16″ and adapted to be positioned closely adjacent the user's second ear. The first earpiece 15, 15′, 15″ and the second earpiece 16, 16′, 16″ serve as sound transmission members to transmit the monitored surface sound from the sound receiver assembly 11, 11′, 11″ to the first eartip 17, 17′, 17″ and the second eartip 18, 18′, 18″, and further to the user's ears.

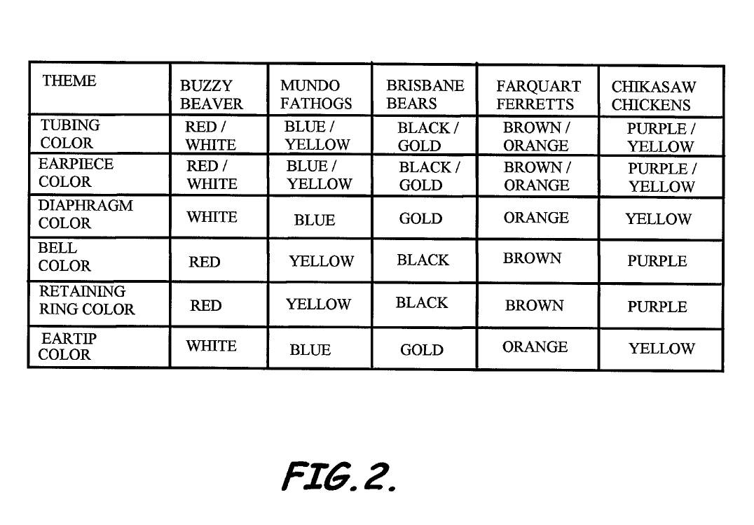

Advantageously the first tube 12, 12′, 12″ preferably is colored with a first color associated with a sports team, which is defined as a first sports team color, while the second tube 13, 13′, 13″ is colored with a second contrasting color also associated with the sports team, which is defined as a second sports team color. The tube retainer 14, 14′, 14″ has sports indicia associated with the same sports team advantageously positioned thereon. In the present invention, the sports indicia have colors corresponding to the first and second sports team colors and may include sports logos, and other sports related indicia readily recognizable by both the user and the patient. Representative examples of the sports indicia include college, professional, club or other sports teams' logos. As illustrated in FIG. 1, one of the stethoscopes 10 preferably is associated with the Buzzy Beavers. The first tube 12 and second tube 13 each advantageously displays one of the two contrasting colors, i.e., red and white (see FIG. 2). The corresponding character is displayed on the tube retainer 14. Another stethoscope 10′ is associated with the Brisbane Bears. The first tube 12′ and second tube 13′ each advantageously displays one of the two contrasting colors, i.e., black and gold (see FIG. 2). The corresponding character is displayed on the tube retainer 14′. The stethoscope 10″ is associated with the Mundo Fathogs. The first tube 12″ and second tube 13″ each advantageously displays one of the two contrasting colors, i.e., blue and yellow (see FIG. 2). The corresponding character is displayed on the tube retainer 14″.

FIG. 2 demonstrates five exemplary sports indicia and corresponding color-coding of various parts of the stethoscope. Three of the five indicia are shown in FIG. 1.

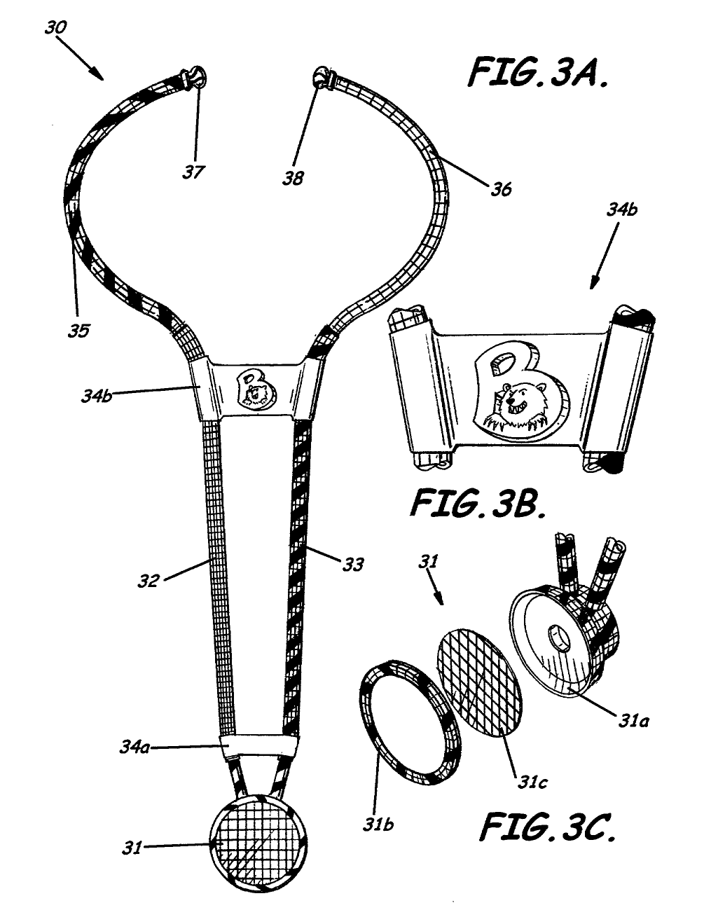

As perhaps best illustrated in FIGS. 3A-3C, the dual channel stethoscope 30 has a sound receiver assembly 31, first and second tubes 32, 33, two tube retainers 34a, 34b, first and second earpieces 35, 36, and first and second eartips 37, 38 (see FIG. 3A). The sound receiver assembly 31 has a bell 31a, a retaining ring 31b mounted to the bell 31a, and a transducer diaphragm 31c mounted to the bell 31a and retained in position by the retaining ring 31b (see FIG. 3C for exploded view).

Two contrasting colors associated with the Brisbane Bears, i.e., black and gold, are advantageously displayed on the first and second tubes 32 and 33, the first and second earpieces 35, 36, and the first and second eartips 37, 38. As illustrated in FIG. 3A, the first tube 32 displays gold color, while the second tube 33 displays black color. The first earpiece 35 displays black color, while the second earpiece 36 displays gold color. The first and second eartips 37 and 38 may also display at least one of the two contrasting colors. The colors can be side-by-side, split, swirled, checkered, striped or other various configurations as understood by those skilled in the art.

Still preferably, the character associated with Brisbane Bears can be advantageously positioned on the tube retainer 34b (see FIG. 3B). Various parts of the sound receiver assembly 31 can also be advantageously color-coded with at least one of the two contrasting colors. For example, the bell 31a and the retaining ring 31b display black color, while the transducer diaphragm 31c displays gold color (see FIG. 3C).

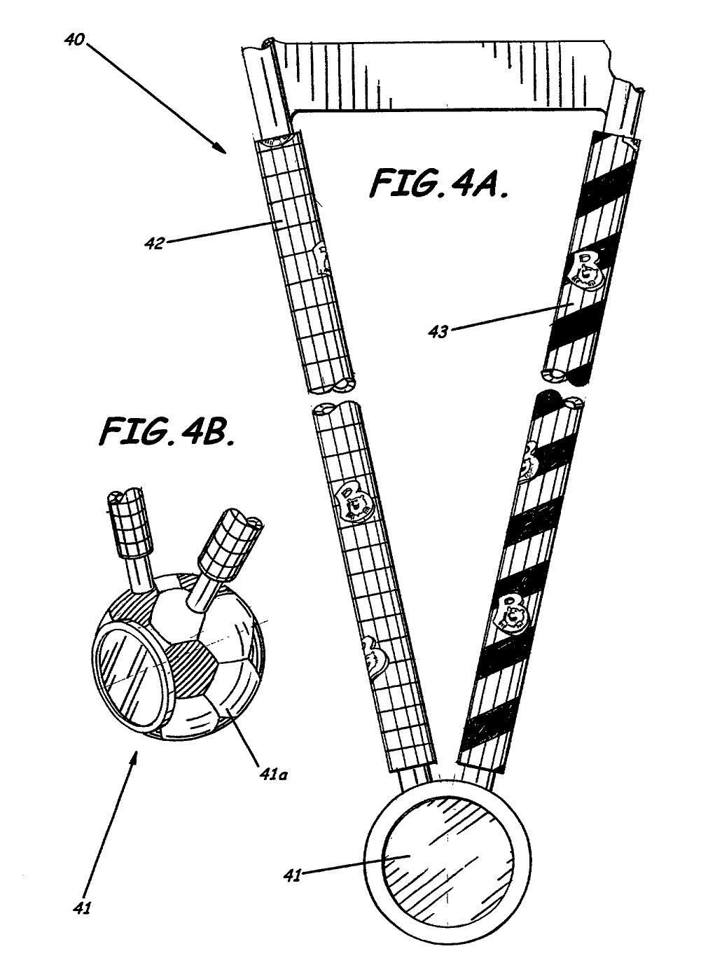

Still preferably, the sports indicia can also be advantageously displayed directly on the first and second tubes. As illustrated in FIG. 4A, the dual channel stethoscope assembly 40 has a first tube 42 and a second tube 43. Each tube has sports indicia positioned thereon in addition to displaying one of the two contrasting colors. Still preferably, the bell 41a can have a shape substantially similar to a ball associated with the sports team, such as a soccer ball (see FIG. 4B).

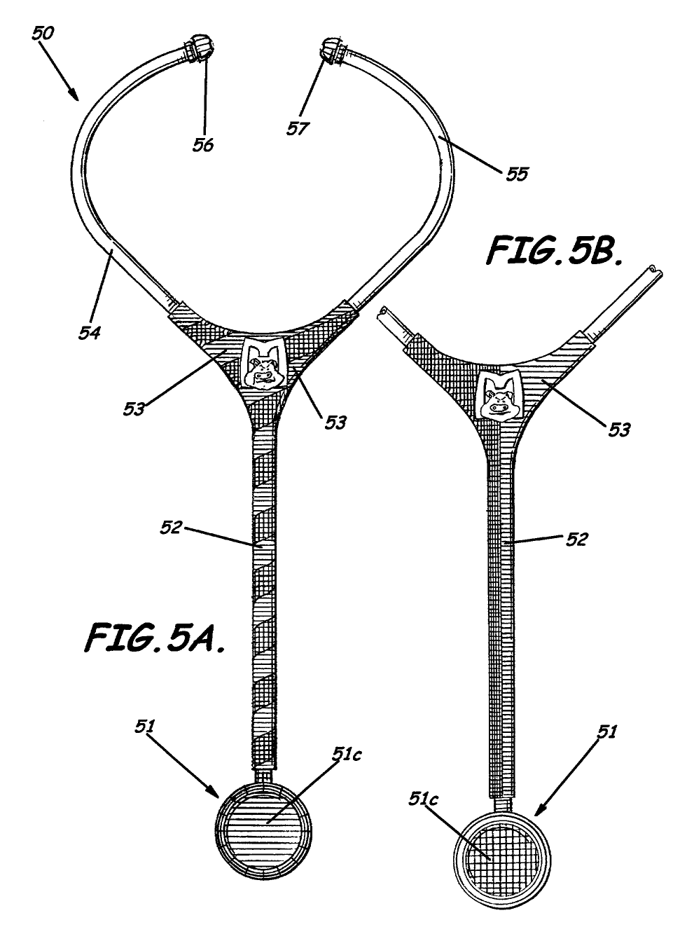

FIGS. 5A and 5B illustrate single channel stethoscope assemblies for sports enthusiasts according to another embodiment of the present invention. Such sports stethoscopes are used for monitoring sound by a user on a body surface of a patient when a proximal portion thereof is positioned closely adjacent the patient and a distal portion is positioned closely adjacent the user. As illustrated in FIG. 5A, a single channel stethoscope assembly 50 preferably has a sound receiver assembly 51, a tube 52 having a proximal end portion connected to and having hollow portions in fluid communication with the sound receiver assembly 51 and extending outwardly toward the user and a manifold 53 connected to a distal end portion of the tube 52, in fluid communication with the hollow portions of the tube 52, and extending outwardly from the distal end portion of the tube 52. A manifold can be two or more passageways, pipes, or hollow portions connecting to another hollow portion to all fluid, e.g., a gas or air, to pass therethrough. The manifold 53 preferably includes at least a first and second hollow portions in fluid communication with the hollow portions of the tube 52. The stethoscope also preferably has a first earpiece 54 having a proximal end portion connected to the manifold 53, in fluid communication with the at least a first hollow portion of the manifold 53, and extending outwardly toward the user, a second earpiece 55 having a proximal end portion connected to the manifold 53, in fluid communication with the at least a second hollow portion of the manifold 53, and extending outwardly toward the user, a first eartip 56 connected to a distal end portion of the first earpiece 54 and adapted to be positioned closely adjacent the user's first ear, and a second eartip 57 connected to a distal end portion of the second earpiece 55 and adapted to be positioned closely adjacent the user's second ear.

The tube 52 and the manifold 53 each is advantageously colored with the two contrasting colors which are associated with the Mundo Fathogs. The two contrasting colors, i.e., blue and yellow, are displayed on the tube 52 and manifold 53 swirled (see FIG. 5A) or striped (see FIG. 5B). At least one of the two contrasting colors, e.g. blue, can also be displayed on the transducer diaphragm 51c.

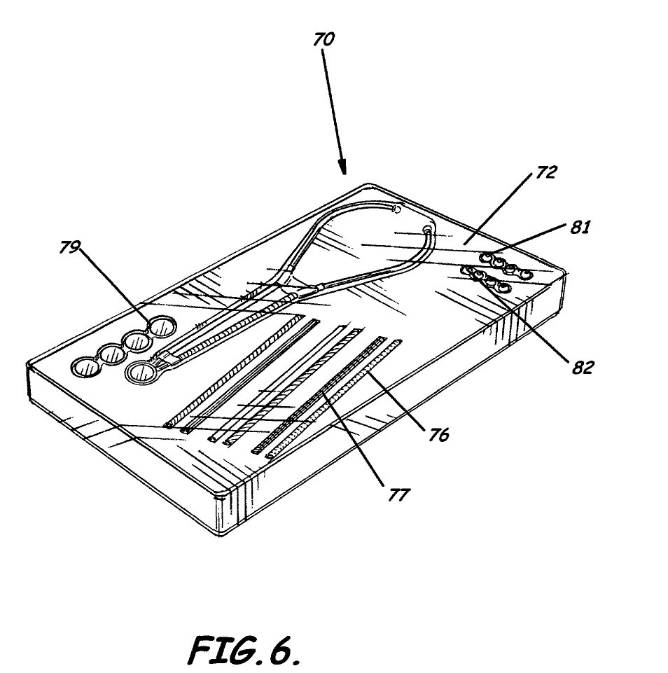

An existing stethoscope can be converted to a sports stethoscope. FIG. 6 illustrates a kit 70 for converting an existing dual channel stethoscope to a sports dual channel stethoscope. Such kit preferably has a container 72, at least first and second tubes 76, 77 each positioned in the container and visibly colored with a first color associated with a sports team to thereby define a first sports team color and with a second contrasting color also associated with the sports team to thereby define a second sports team color, at least first and second eartips 81, 82 each positioned in the container and having at least one of the first and second sports team colors, and at least one transducer diaphragm 79 positioned in the container and having a surface visibly colored with at least one of the first and second sports team colors.

The present invention further advantageously provides a method of retrofitting an existing stethoscope for a sports stethoscope. Such method preferably includes replacing a tube assembly of the existing stethoscope with a different tube assembly being visibly colored with at least one of a first color associated with a sports team to thereby define a first sports team color and a second contrasting color also associated with the sports team to thereby define a second sports team color.

Alternatively, the retrofitting method can further include replacing first and second eartips of the existing stethoscope with third and fourth eartips. Each of the third and fourth eartips preferably is visibly colored with at least one of the first and second sports team colors.

Still alternatively, the retrofitting method can further include replacing first and second earpieces of the existing stethoscope with third and fourth earpieces. Each of the third and fourth earpieces preferably is visibly colored with at least one of the first and second sports team colors.

Still alternatively, the retrofitting method can further include replacing at least one transducer diaphragm of the existing stethoscope with at least one transducer diaphragm being visibly colored with at least one of the first and second sports team colors.

To further target sports teams, the method can yet further include positioning sports indicia on at least one transducer diaphragm of the existing stethoscope, wherein the sports indicia have colors corresponding to the first and second sports team colors associated with the tube assembly. Further more, the method can include positioning the sports indicia on the tube assembly visibly colored with at least one of the first and second sports team colors. In the present invention, the sports indicia have colors corresponding to the first and second sports team colors and may include sports logos, and other sports related indicia readily recognizable by both the user and the patient. For dual channel stethoscope, the sports indicia may also be positioned on at least one tube retainer of the existing stethoscope.

According to yet another embodiment of the present invention, a method of doing business is advantageously provided by obtaining a license from at least one sports teams to use sports team identifiers associated with the sports team in association with a stethoscope, positioning sports team identifiers on a stethoscope so that a potential user of the stethoscope readily recognizes that the stethoscope is associated with the sports team, selling the stethoscope to the potential user, and distributing the stethoscope to the potential user after purchase of the stethoscope. Such method can further include advertising the stethoscope associated with the sports team.

Preferably, the sports team can include at least one of the following: a school sports team, a professional sports team and a club sports team. Still preferably, the sports identifier is positioned on at least one of the following: a tube assembly, first and second eartips, first and second earpieces, and a sound receiver assembly of the stethoscope.

Another method of doing business according to the present invention preferably includes obtaining a license from each of a plurality of different sports teams to use sports team identifiers associated with each of the plurality of sports teams in association with a plurality of stethoscopes; and manufacturing a plurality of stethoscopes. Each of the plurality of stethoscopes preferably has a different sports team identifier associated therewith. Such method can further include advertising each of the plurality of stethoscopes associated with each of the plurality of sports teams or selling the plurality of stethoscopes to the potential user and distributing the plurality of stethoscopes to the potential user after purchase of the stethoscopes.

Preferably, the sports team can include at least one of the following: a school sports team, a professional sports team and a club sports team. Still preferably, the sports identifier is positioned on at least one of the following: a tube assembly, first and second eartips, first and second earpieces, and a sound receiver assembly of the stethoscope.

In the drawings and specification, there have been disclosed a typical preferred embodiment of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

That claimed is:

1. A dual channel stethoscope assembly for sports enthusiasts for monitoring sound by a user on a body surface of a patient when a proximal portion thereof is positioned closely adjacent the patient and a distal portion is positioned closely adjacent the user, the stethoscope comprising:
(a) at least one sound receiver assembly comprising a bell, a retaining ring mounted to the bell, and a transducer diaphragm mounted to the bell, retained in position by the retaining ring, and being adapted to abuttingly contact the body surface to thereby receive sound therefrom, the transducer diaphragm further having a surface visibly colored with a first color associated with a sports team to thereby define a first sports team color;
(b) first and second tubes each having a proximal end portion connected to the at least one sound receiver assembly and extending outwardly therefrom toward the user, the first and second tubes being visibly colored with the first sports team color and a second contrasting color also associated with the sports team to thereby define a second sports team color;
(c) at least one tube retainer retaining at least a portion of each of the first and second tubes in a position closely adjacent each other;
(d) a first earpiece having a proximal end portion connected to a distal end portion of the first tube and extending outwardly therefrom toward the user;
(e) a second earpiece having a proximal end portion connected to a distal end portion of the second tube and extending outwardly therefrom toward the user;
(f) a first eartip connected to a distal end portion of the first earpiece and adapted to be positioned closely adjacent the user's first ear, the first eartip having at least one of the first and second sports team colors associated therewith; and
(g) a second eartip connected to a distal end portion of the second earpiece and adapted to be positioned closely adjacent the user's second ear, the second eartip also having at least one of the first and second sports team colors associated therewith.

2. The dual channel stethoscope assembly of claim 1, wherein the sports team includes at least one of the following: a school sports team, a professional sports team, and a club sports team.

3. The dual channel stethoscope assembly of claim 1, wherein the first tube is colored with the first sports team color, and wherein the second tube is colored with the second sports team color.

4. The dual channel stethoscope assembly of claim 1, wherein the first and second tubes further have sports indicia positioned thereon and readily visible and recognizable to the user and the patient.

5. The dual channel stethoscope assembly of claim 1, wherein the first eartip is colored with the first sports team color, and wherein the second eartip is colored with the second sports team color.

6. The dual channel stethoscope assembly of claim 1, wherein the first earpiece is colored with the first sports team color, and wherein the second earpiece is colored with the second sports team color.

7. The dual channel stethoscope assembly of claim 1, wherein the bell is colored with at least one of the first and second sports team colors, and wherein the retaining ring is colored with at least one of the first and second sports team colors.

8. The dual channel stethoscope assembly of claim 1, wherein the transducer diaphragm further has sports indicia positioned thereon and readily visible and recognizable to the user and the patient, the sports indicia having colors corresponding to the first and second sports team colors associated therewith.

9. The dual channel stethoscope assembly of claim 1, wherein the at least one tube retainer has sports indicia readily visible and recognizable to the user and the patient positioned thereon.

10. The dual channel stethoscope assembly of claim 1, wherein the bell has a shape substantially similar to a ball associated with the sports team.

11. A dual channel stethoscope assembly for sports enthusiasts for monitoring sound by a user on a body surface of a patient when a proximal portion thereof is positioned closely adjacent the patient and a distal portion is positioned closely adjacent the user, the stethoscope comprising:
(a) at least one sound receiver assembly comprising a bell, a retaining ring mounted to the bell, and a transducer diaphragm mounted to the bell, retained in position by the retaining ring, and being adapted to abuttingly contact the body surface to thereby receive sound therefrom;
(b) first and second tubes each having a proximal end portion connected to the at least one sound receiver assembly and extending outwardly therefrom toward the user, the first and second tubes being visibly colored with a first color associated with a sports team to thereby define a first sports team color and a second contrasting color also associated with the sports team to thereby define a second sports team color;
(c) at least one tube retainer retaining at least a portion of each of the first and second tubes in a position closely adjacent each other;
(d) a first earpiece having a proximal end portion connected to a distal end portion of the first tube and extending outwardly therefrom toward the user;
(e) a second earpiece having a proximal end portion connected to a distal end portion of the second tube and extending outwardly therefrom toward the user;
(f) a first eartip connected to a distal end portion of the first earpiece and adapted to be positioned closely adjacent the user's first ear; and
(g) a second eartip connected to a distal end portion of the second earpiece and adapted to be positioned closely adjacent the user's second ear.

12. The dual channel stethoscope assembly of claim 11, wherein the sports team includes at least one of the following: a school sports team, a professional sports team, and a club sports team.

13. The dual channel stethoscope assembly of claim 11, wherein the first tube is colored with the first sports team color, and wherein the second tube is colored with the second sports team color.

14. The dual channel stethoscope assembly of claim 11, wherein the first and second tubes further have sports indicia positioned thereon and readily visible and recognizable to the user and the patient.

15. The dual channel stethoscope assembly of claim 11, wherein the first eartip is colored with the first sports team color, and wherein the second eartip is colored with the second sports team color.

16. The dual channel stethoscope assembly of claim 11, wherein the first earpiece is colored with the first sports team color, and wherein the second earpiece is colored with the second sports team color.

17. The dual channel stethoscope assembly of claim 11, wherein the bell is colored with at least one of the first and second sports team colors, and wherein the retaining ring is colored with at least one of the first and second sports team colors.

18. The dual channel stethoscope assembly of claim 11, wherein the transducer diaphragm further having a surface visibly colored with at least one of the first and second sports team colors.

19. The dual channel stethoscope assembly of claim 11, wherein the transducer diaphragm further has sports indicia positioned thereon and readily visible and recognizable to the user and the patient, the sports indicia having colors corresponding to the first and second sports team colors associated therewith.

20. The dual channel stethoscope assembly of claim 11, wherein the at least one tube retainer has sports indicia readily visible and recognizable to the user and the patient positioned thereon.

21. The dual channel stethoscope assembly of claim 11, wherein the bell has a shape substantially similar to a ball associated with the sports team.

22. A single channel stethoscope assembly for sports enthusiasts for monitoring sound by a user on a body surface of a patient when a proximal portion thereof is positioned closely adjacent the patient and a distal portion is positioned closely adjacent the user, the stethoscope comprising:
(a) at least one sound receiver assembly comprising a bell, a retaining ring mounted to the bell, and a transducer diaphragm mounted to the bell, retained in position by the retaining ring, and being adapted to abuttingly contact the body surface to thereby receive sound therefrom, the transducer diaphragm further having a surface visibly colored with a first color associated with a sports team to thereby define a first sports team color;
(b) a tube having a proximal end portion connected to the at least one sound receiver assembly, having hollow portions in fluid communication with the bell and extending outwardly from the at least one sound receive assembly toward the user, the tube being visibly colored with the first sports team color and a second contrasting color also associated with the sports team to thereby define a second sports team color;
(c) a manifold connected to a distal end portion of the tube, in fluid communication with the hollow portions of the tube, and extending outwardly from the distal end portion of the tube, the manifold including at least a first hollow portion in fluid communication with the hollow portions of the tube and at least a second hollow portion also in fluid communication with the hollow portions of the tube, the manifold being visibly colored with at least one of the first and second sports team colors;
(d) a first earpiece having a proximal end portion connected to the manifold, in fluid communication with the at least a first hollow portion of the manifold, and extending outwardly toward the user;
(e) a second earpiece having a proximal end portion connected to the manifold, in fluid communication with the at least a second hollow portion of the manifold, and extending outwardly toward the user;
(f) a first eartip connected to a distal end portion of the first earpiece and adapted to be positioned closely adjacent the user's first ear, the first eartip having at least one of the first and second sports team colors associated therewith; and
(g) a second eartip connected to a distal end portion of the second earpiece and adapted to be positioned closely adjacent the user's second ear, the second eartip also having at least one of the first and second sports team colors associated therewith.

23. The single channel stethoscope assembly of claim 22, wherein the sports team includes at least one of the following: a school sports team, a professional sports team, and a club sports team.

24. The single channel stethoscope assembly of claim 22, wherein the tube further has a first colored portion and a second colored portion.

25. The single channel stethoscope assembly of claim 22, wherein the manifold further has a first colored portion and a second colored portion.

26. The single channel stethoscope assembly of claim 22, wherein the manifold further has sports indicia positioned thereon and readily visible and recognizable to the user and the patient.

27. The single channel stethoscope assembly of claim 22, wherein the first eartip is colored with the first sports team color, and wherein the second eartip is colored with the second sports team color.

28. The single channel stethoscope assembly of claim 22, wherein the first earpiece is colored with the first sports team color, and wherein the second earpiece is colored with the second sports team color.

29. The single channel stethoscope assembly of claim 22, wherein the bell is colored with at least one of the first and second sports team colors, and wherein the retaining ring is colored with at least one of the first and second sports team colors.

30. The single channel stethoscope assembly of claim 22, wherein the transducer diaphragm further has sports indicia positioned thereon and readily visible and recognizable to the user and the patient, the sports indicia having colors corresponding to the first and second sports team colors associated therewith.

31. The single channel stethoscope assembly of claim 22, wherein the bell has a shape substantially similar to a ball associated with the sports team.

32. A single channel stethoscope assembly for sports enthusiasts for monitoring sound by a user on a body surface of a patient when a proximal portion thereof is positioned closely adjacent the patient and a distal portion is positioned closely adjacent the user, the stethoscope comprising:
(a) at least one sound receiver assembly comprising a bell, a retaining ring mounted to the bell, and a transducer diaphragm mounted to the bell, retained in position by the retaining ring, and being adapted to abuttingly contact the body surface to thereby receive sound therefrom;
(b) a tube having a proximal end portion connected to the at least one sound receiver assembly, having hollow portions in fluid communication with the bell and extending outwardly from the at least one sound receive assembly toward the user, the tube being visibly colored with a first color associated with a sports team to thereby define a first sports team color and a second contrasting color also associated with the sports team to thereby define a second sports team color;
(c) a manifold connected to a distal end portion of the tube, in fluid communication with the hollow portions of the tube, and extending outwardly from the distal end portion of the tube, the manifold including at least a first hollow portion in fluid communication with the hollow portions of the tube and at least a second hollow portion also in fluid communication with the hollow portions of the tube, the manifold being visibly colored with at least one of the first and second sports team colors;
(d) a first earpiece having a proximal end portion connected to the manifold, in fluid communication with the at least a first hollow portion of the manifold, and extending outwardly toward the user;
(e) a second earpiece having a proximal end portion connected to the manifold, in fluid communication with the at least a second hollow portion of the manifold, and extending outwardly toward the user;
(f) a first eartip connected to a distal end portion of the first earpiece and adapted to be positioned closely adjacent the user's first ear; and
(g) a second eartip connected to a distal end portion of the second earpiece and adapted to be positioned closely adjacent the user's second ear.

33. The single channel stethoscope assembly of claim 32, wherein the sports team includes at least one of the following: a school sports team, a professional sports team, and a club sports team.

34. The single channel stethoscope assembly of claim 32, wherein the tube further has a first colored portion and a second colored portion.

35. The single channel stethoscope assembly of claim 32, wherein the manifold further has a first colored portion and a second colored portion.

36. The single channel stethoscope assembly of claim 32, wherein the manifold further has sports indicia positioned thereon and readily visible and recognizable to the user and the patient.

37. The single channel stethoscope assembly of claim 32, wherein the first eartip is colored with the first sports team color, and wherein the second eartip is colored with the second sports team color.

38. The single channel stethoscope assembly of claim 32, wherein the first earpiece is colored with the first sports team color, and wherein the second earpiece is colored with the second sports team color.

39. The single channel stethoscope assembly of claim 32, wherein the bell is colored with at least one of the first and second sports team colors, and wherein the retaining ring is colored with at least one of the first and second sports team colors.

40. The single channel stethoscope assembly of claim 32, wherein the transducer diaphragm further having a surface visibly colored with at least one of the first and second sports team colors.

41. The single channel stethoscope assembly of claim 32, wherein the transducer diaphragm further has sports indicia positioned thereon and readily visible and recognizable to the user and the patient, the sports indicia having colors corresponding to the first and second sports team colors associated therewith.

42. The single channel stethoscope assembly of claim 32, wherein the bell has a shape substantially similar to a ball associated with the sports team.

43. A stethoscope assembly for sports enthusiasts for monitoring sound by a user on a body surface of a patient when a proximal portion thereof is positioned closely adjacent the patient and a distal portion is positioned closely adjacent the user, the stethoscope comprising:
(a) at least one sound receiver assembly comprising a bell, a retaining ring mounted to the bell, and a transducer diaphragm mounted to the bell, retained in position by the retaining ring, and being adapted to abuttingly contact the body surface to thereby receive sound therefrom;
(b) a tube assembly having a proximal end portion connected to the at least one sound receiver assembly and extending outwardly therefrom toward the user, the tube assembly further being visibly colored with at least one of a first color associated with a sports team to thereby define a first sports team color and a second contrasting color also associated with the sports team to thereby define a second sports team color;
(c) a first earpiece having a proximal end portion connected to a distal end portion of the tube assembly and extending outwardly therefrom toward the user; and
(d) a second earpiece having a proximal end portion connected to the distal end portion of the tube assembly and extending outwardly therefrom toward the user.

44. The stethoscope of claim 43, wherein the sports team includes at least one of the following: a school sports team, a professional sports team, and a club sports team.

45. The stethoscope of claim 43, wherein the tube assembly further has sports indicia positioned thereon and readily visible and recognizable to the user and patient.

46. The stethoscope of claim 43, wherein the first earpiece is colored with the first sports team color, and wherein the second earpiece is colored with the second sports team color.

47. The stethoscope of claim 43, wherein the bell is visibly colored with at least one of the first and second sports team colors, and wherein the retaining ring is colored with at least one of the first and second sports team colors.

48. The stethoscope of claim 43, wherein the transducer diaphragm further having a surface visibly colored with at least one of the first and second sports team colors.

49. The stethoscope of claim 43, wherein the transducer diaphragm further has sports indicia positioned thereon and readily visible and recognizable to the user and patient, the sports indicia having colors corresponding to the first and second sports team colors associated therewith.

50. A kit for converting an existing dual channel stethoscope to a sports enthusiast's dual channel stethoscope, the kit comprising:
(a) a container;
(b) at least first and second tubes each positioned in the container and visibly colored with a first color associated with a sports team to thereby define a first sports team color and with a second contrasting color also associated with the sports team to thereby define a second sports team color;
(c) at least first and second eartips each positioned in the container and having at least one of the first and second sports team colors; and
(d) at least one transducer diaphragm positioned in the container and having a surface visibly colored with at least one of the first and second sports team colors.

51. The kit of claim 50, wherein the sports team includes at least one of the following: a school sports team, a professional sports team, and a club sports team.

52. The kit of claim 50, wherein the first tube is colored with the first sports team color, and wherein the second tube is colored with the second sports team color.

53. The kit of claim 50, wherein the first and second tubes further have sports indicia positioned thereon and readily visible and recognizable to a user and a patient.

54. The kit of claim 50, wherein the first eartip is colored with the first sports team color, and wherein the second eartip is colored with the second sports team color.

55. The kit of claim 50, wherein the at least one transducer diaphragm further has sports indicia positioned thereon and readily visible and recognizable to the user and the patient, the sports indicia having colors corresponding to the first and second sports team colors associated therewith.

56. A kit for converting an existing single channel stethoscope to a sports enthusiast's single channel stethoscope, the kit comprising:
(a) a container;
(b) at least one tube positioned in the container and being visibly colored with a first color associated with a sports team to thereby define a first sports team color and with a second contrasting color also associated with the sports team to thereby define a second sports team color;
(c) at least one manifold positioned in the container and being visibly colored with at least one of the first and second sports team colors;
(d) at least first and second eartips positioned in the container and each having at least one of the first and second sports team colors associated therewith; and
(e) at least one transducer diaphragm positioned in the container and having a surface visibly colored with at least one of the first and second sports team colors.

57. The kit of claim 56, wherein the sports team includes at least one of the following: a school sports team, a professional sports team, and a club sports team.

58. The kit of claim 56, wherein the at least one tube further has a first colored portion and a second colored portion.

59. The kit of claim 56, wherein the at least one manifold further has a first colored portion and a second colored portion.

60. The kit of claim 56, wherein the manifold further has sports indicia positioned thereon and readily visible and recognizable to the user and the patient positioned thereon.

61. The kit of claim 56, wherein the first eartip is colored with the first sports team color, and wherein the second eartip is colored with the second sports team color.

62. The kit of claim 56, wherein the at least one transducer diaphragm has sports indicia readily visible and recognizable to the user and the patient positioned thereon, the sports indicia having colors corresponding to the first and second sports team colors associated therewith.

63. A kit for converting an existing stethoscope to a sports enthusiast's stethoscope, the kit comprising:
(a) a container;
(b) a tube assembly positioned in the container and being visibly colored with a first color associated with a sports team to thereby define a first sports team color and with a second contrasting color also associated with the sports team to thereby define a second sports team color; and
(c) at least first and second eartips positioned in the container and each having at least one of the first and second sports team colors associated therewith.

64. The kit of claim 63, further comprising:
at least one transducer diaphragm positioned in the container and having a surface visibly colored with at least one of the first and second sports team colors.

65. The kit of claim 63, wherein the sports team includes at least one of the following: a school sports team, a professional sports team, and a club sports team.

66. The kit of claim 63, wherein the tube assembly further has sports indicia positioned thereon and readily visible and recognizable to the user and the patient positioned thereon.

67. The kit of claim 63, wherein the first eartip is colored with the first sports team color, and wherein the second eartip is colored with the second sports team color.

68. The kit of claim 63, wherein the at least one transducer diaphragm has sports indicia readily visible and recognizable to the user and the patient positioned thereon, the sports indicia having colors corresponding to the first and second sports team colors associated therewith.

69. A method of retrofitting an existing stethoscope for sports enthusiasts, the method comprising:
replacing a tube assembly of the existing stethoscope with a different tube assembly being visibly colored with a first color associated with a sports team to thereby define a first sports team color and a second contrasting color also associated with the sports team to thereby define a second sports team color.

70. The method of claim 69, further comprising:
replacing first and second eartips of the existing stethoscope with third and fourth eartips, each of the third and fourth eartips being visibly colored with at least one of the first and second sports team colors.

71. The method of claim 69, further comprising:
replacing first and second earpieces of the existing stethoscope with third and fourth earpieces, each of the third and fourth earpieces being visibly colored with at least one of the first and second sports team colors.

72. The method of claim 69, further comprising:
replacing at least one transducer diaphragm of the existing stethoscope with at least one transducer diaphragm being visibly colored with at least one of the first and second sports team colors.

73. The method of claim 69, further comprising:
positioning sports indicia on at least one transducer diaphragm of the existing stethoscope, the sports indicia having colors corresponding to the first and second sports team colors associated with the tube assembly.

74. The method of claim 69, wherein the existing stethoscope is a dual channel stethoscope.

75. The method of claim 74, further comprising:
positioning sports indicia on at least one tube retainer of the existing stethoscope, the sports indicia having colors corresponding to the first and second sports team colors associated with.

76. The method of claim 69, further comprising:
positioning sports indicia on the tube assembly visibly colored with at least one of the first and second sports team colors, the sports indicia having colors corresponding to the first and second sports team colors associated therewith.

77. A method of doing business, the method comprising:
obtaining a license from at least one sports teams to use sports team identifiers associated with the sports team in association with a stethoscope;
positioning sports team identifiers on a stethoscope so that a potential user of the stethoscope readily recognizes that the stethoscope is associated with sports team;
selling the stethoscope to the potential user; and
distributing the stethoscope to the potential user after purchase of the stethoscope.

78. The method of claim 77, further comprising:
advertising the stethoscope associated with the sports team.

79. The method of claim 77, wherein the sports team includes at least one of the following: a school sports team, a professional sports team, and a club sports team.

80. The method of claim 77, wherein the sports identifier is positioned on at least one of the following: a tube assembly, first and second eartips, first and second earpieces, and a sound receiver assembly of the stethoscope.

81. A method of doing business, the method comprising:
obtaining a license from each of a plurality of different sports teams to use sports team identifiers associated with each of the plurality of sports teams in association with a plurality of stethoscopes; and
manufacturing a plurality of stethoscopes, each stethoscope having a different sports team identifier associated therewith.

82. The method of claim 81, further comprising:
advertising each of the plurality of stethoscopes associated with each of the plurality of sports teams.

83. The method of claim 81, further comprising:
selling the plurality of stethoscopes to the potential user; and
distributing the plurality of stethoscopes to the potential user after purchase of the stethoscopes.

84. The method of claim 81, wherein the sports team includes at least one of the following: a school sports team, a professional sports team, and a club sports team.

85. The method of claim 81, wherein the sports identifier is positioned on at least one of the following: a tube assembly, first and second eartips, first and second earpieces, and a sound receiver assembly of each of the plurality of stethoscopes.
